(19) United States
(12) Patent Application Publication
(10) Pub. No.: US 2024/0201161 A1
(43) Pub. Date: Jun. 20, 2024

(54) CORROSION ESTIMATION DEVICE AND METHOD

(71) Applicant: Nippon Telegraph and Telephone Corporation, Tokyo (JP)

(72) Inventors: Shingo Mineta, Tokyo (JP); Shota Oki, Tokyo (JP); Mamoru Mizunuma, Tokyo (JP); Soichi Oka, Tokyo (JP)

(21) Appl. No.: 18/555,806

(22) PCT Filed: May 21, 2021
(86) PCT No.: PCT/JP2021/019333

§ 371 Date: Oct. 17, 2023

(51) Int. Cl. G01N 33/24; G01N 17/02

(52) U.S. Cl. CPC G01N 33/24; G01N 17/02

(57) ABSTRACT

A first estimation function unit estimates, on the basis of a particle diameter of soil of a target land, a relationship between an underground depth of the land and an oxygen concentration in soil. A second estimation function unit estimates, on the basis of a corrosion rate or a corrosion amount of a target metal near a ground surface of the land and the relationship estimated by the first estimation function unit, a relationship between the underground depth and the corrosion rate or the corrosion amount in the land from a relationship between the oxygen concentration and the corrosion rate or the corrosion amount. A third estimation function unit estimates a corrosion state of a structure constituted of the metal buried in target ground in the land from the relationship estimated by the second estimation function unit.

### CROSS-REFERENCE TO RELATED APPLICATIONS

This application is a national phase entry of PCT Application No. PCT/JP2021/019333, filed on May 21, 2021, which application is hereby incorporated herein by reference.

### TECHNICAL FIELD

The present invention relates to a corrosion estimation device and a corrosion estimation method for estimating corrosion of a structure buried in soil.

### BACKGROUND

There are many types of infrastructure equipment that support our life, and the number of pieces of infrastructure equipment is also enormous. In addition, infrastructure equipment is exposed to various environments not only in urban areas but also in mountainous areas, the vicinity of coasts, hot spring areas, cold areas, and the sea and the ground, and deterioration forms and deterioration progress rates are various. To maintain infrastructure equipment having such characteristics, it is necessary to grasp the current state of deterioration by visual inspection or the like.

As infrastructure equipment, for example, underground equipment made of metal typified by steel pipe columns, support anchors, steel pipes, and the like corrode due to contact with soil, and deteriorate at different speeds depending on the external environment (Non Patent Literature 1, Non Patent Literature 2, and Non Patent Literature 3). However, if a deterioration state is near the ground, it is possible to visually observe or directly measure the deterioration state, but it is not possible to check a deterioration state of a portion hidden by the soil by visual inspection. For this reason, in the underground equipment, it is difficult to perform efficient maintenance depending on the deterioration state.

The following is conceivable as a method of grasping the deterioration state of the underground equipment. For example, a method of monitoring a deterioration state by attaching a sensor or the like before laying target equipment can be considered. Further, a method of embedding a sensor, a metal, or the like to a target depth and acquiring information related to a deterioration state can be considered.

However, the former method causes a cost increase particularly in a case where the number of pieces of target equipment is large, and it is difficult to perform work for a failure or update of the sensor after being embedded. Further, in the latter method, when the target embedding position is deep, it is necessary to embed the target deep in the ground by excavation or the like, and thus there are many cases where it is difficult in terms of cost and technology. As described above, in the conventional methods, it is difficult to easily and inexpensively grasp the deterioration state of underground equipment.

### CITATION LIST

### Non Patent Literature

Non Patent Literature 1: Morio KADOI et al., “Studies on Soil Corrosion of Metallic Materials (Part 1)—Fundamental Experiment on Soils-”, CORROSION ENGINEERING, Vol. 16, No. 6, pp. 238-246, 1967.
Non Patent Literature 2: Yoshikazu Miyata, Shukuji Asakura, “Corrosion Monitoring of Metals in Soils by Electrochemical and Related Methods: Part 2—Estimation of the Corrosion Rate Based upon the Combination of a Number of Informations and Proposals of the Systematic Evaluation Process—”, Zairyo-to-Kankyo, Vol. 46, No. 10, pp. 610-619, 1997.
Non Patent Literature 3: Satomi Tsunoda, Tetsuro Akiba, “Some Problem for Evaluating Soil Aggressivity”, Corrosion Engineering, Vol. 36, No. 3, pp. 168-177, 1987.

### SUMMARY

### Technical Problem

As described above, conventionally, there has been a problem that it is difficult to simply and inexpensively inspect a deterioration state of underground equipment made of metal.

Embodiments of the present invention has been made to solve the above problems, and an object thereof is to enable simple and inexpensive inspection of a deterioration state of underground equipment made of metal.

### Solution to Problem

A corrosion estimation method according to embodiments of the present invention includes a first step of estimating, on the basis of a particle diameter of soil of a target land, a relationship between an underground depth of the land and an oxygen concentration in soil, and a second step of estimating, on the basis of a corrosion rate or a corrosion amount of a target metal near a ground surface of the land and the relationship estimated in the first step, a relationship between the underground depth and the corrosion rate or the corrosion amount in the land from a relationship between the oxygen concentration and the corrosion rate or the corrosion amount.

In addition, a corrosion estimation device according to embodiments of the present invention includes a first estimation function unit that estimates, on the basis of a particle diameter of soil of a target land, a relationship between an underground depth of the land and an oxygen concentration in soil, and a second estimation function unit that estimates, on the basis of a corrosion rate or a corrosion amount of a target metal near a ground surface of the land and the relationship estimated by the first estimation function unit, a relationship between the underground depth and the corrosion rate or the corrosion amount in the land from a relationship between the oxygen concentration and the corrosion rate or the corrosion amount.

### Advantageous Effects of Embodiments of Invention

As described above, according to the present invention, since a relationship between an underground depth in a land and a corrosion rate or a corrosion amount is estimated, a deterioration state of underground equipment made of metal can be easily and inexpensively checked.

### BRIEF DESCRIPTION OF THE DRAWINGS

FIG. 1 is a configuration diagram illustrating a configuration of a corrosion estimation device according to an embodiment of the present invention.

FIG. 2 is a flowchart for explaining a corrosion estimation method according to the embodiment of the present invention.

FIG. 3 is a configuration diagram illustrating a hardware configuration of the corrosion estimation device according to the embodiment of the present invention.

FIG. 4 is an explanatory diagram illustrating an image of estimation using a corrosion estimation device 100 according to the embodiment of the present invention.

FIG. 5 is a distribution diagram illustrating a distribution of particle diameters of soil.

FIG. 6 is a characteristic diagram illustrating a relationship between an underground depth and an oxygen concentration in soil.

FIG. 7 is a characteristic diagram illustrating a relationship between an oxygen concentration and a corrosion rate or a corrosion amount.

FIG. 8 is a characteristic diagram illustrating a relationship between an underground depth and the corrosion rate or the corrosion amount.

### DETAILED DESCRIPTION OF ILLUSTRATIVE EMBODIMENTS

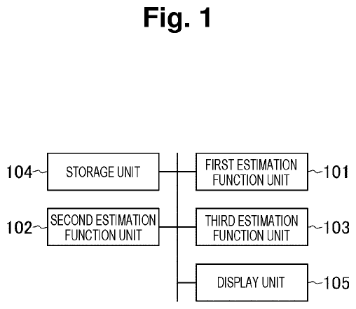

Hereinafter, a corrosion estimation device according to an embodiment of the present invention will be described with reference to FIG. 1. The corrosion estimation device includes a first estimation function unit 101, a second estimation function unit 102, a storage unit 104, and a display unit 105.

The first estimation function unit 101 estimates the relationship between an underground depth of a land and an oxygen concentration in soil on the basis of the particle diameter of the soil of a target land. The second estimation function unit 102 estimates the relationship between the underground depth and a corrosion rate or a corrosion amount in the land from the relationship between an oxygen concentration and a corrosion rate or a corrosion amount on the basis of the corrosion rate or the corrosion amount of the target metal near a ground surface of the land and the relationship estimated by the first estimation function unit 101.

For example, the corrosion rate or the corrosion amount of the target metal near the ground surface of the target land and the particle diameter of the soil of the land can be acquired in advance and stored in the storage unit 104. Further, the relationship between the oxygen concentration and the corrosion rate or the corrosion amount can also be stored in the storage unit 104.

In addition, the corrosion estimation device according to the embodiment includes a third estimation function unit 103 that estimates the corrosion state of a structure constituted of a metal buried in the target ground in the land from the relationship estimated by the second estimation function unit 102. The estimated corrosion state is displayed on the display unit 105, for example.

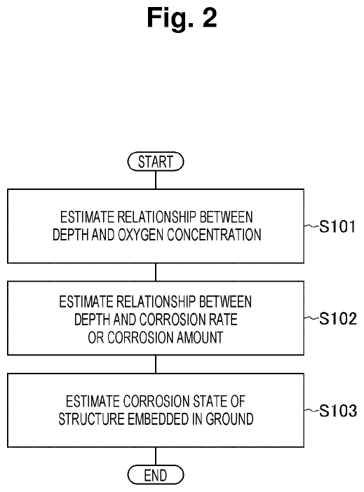

Next, a corrosion estimation method according to an embodiment of the present invention will be described with reference to FIG. 2.

First, in a first step S101, the first estimation function unit 101 estimates the relationship between the underground depth of the land and the oxygen concentration in the soil on the basis of the particle diameter of the soil of the target land.

Next, in a second step S102, the second estimation function unit 102 estimates the relationship between the underground depth and the corrosion rate or the corrosion amount in the land from the relationship between the oxygen concentration and the corrosion rate or the corrosion amount on the basis of the corrosion rate or the corrosion amount of the target metal near the ground surface of the land and the relationship estimated in the first step S101.

Next, in a third step S103, from the relationship estimated in the second step S102, the corrosion state of the structure constituted of the metal buried in the target ground in the land is estimated.

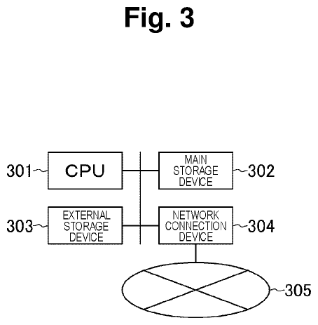

Note that, as illustrated in FIG. 3, the corrosion estimation device according to the above-described embodiment can implement the above-described functions (corrosion estimation method) by forming a computer device including a central processing unit (CPU) 301, a main storage device 302, an external storage device 303, a network connection device 304, and the like, and causing the CPU 301 to be operated by a program developed in the main storage device 302 (execute a program). The program is a program for a computer to execute the corrosion estimation method described in the above embodiment. The network connection device 304 is connected to a network 305. Further, the functions can be distributed to a plurality of computer devices.

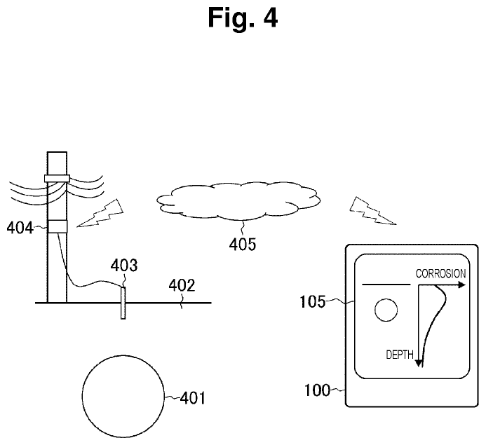

Here, as illustrated in FIG. 4, the corrosion rate or the corrosion amount described above can be obtained by a measurement unit 403 constituted of a sensor, a metal, or the like installed in equipment 401 which is a structure constituted of metal to be estimated or near a ground surface 402 close to the equipment. For example, the corrosion amount can be acquired by actually measuring the corrosion amount at a portion of the equipment 401 close to the ground using the measurement unit 403. In addition, the corrosion rate can be obtained by dividing the measured corrosion amount at a place close to the ground by the age of the equipment. The corrosion rate can also be acquired from a sensor or a metal installed close to the ground near the target equipment.

The measurement unit 403 can be constituted of a sensor or a metal installed close to the equipment 401 or the ground surface 402 near the equipment 401. For example, in a case where the corrosion amount near the ground (ground surface 402) of the equipment 401 itself is measured, this is the measurement unit 403. Further, a sensor, a metal, or the like can be installed as the measurement unit 403 near the ground of the equipment 401. Furthermore, the measurement unit 403 can be installed near the ground surface 402 of the target place away from the equipment 401. However, in this case, it is preferable to install the measurement unit 403 in an environment as close as possible to the installation environment of the equipment 401, and accuracy is enhanced by installing the measurement unit 403 as close as possible to the equipment 401.

The measurement unit 403 can acquire information related to the corrosion rate or the corrosion amount, and in general, it is preferable to use a measurement unit including the same type of material as the metal to be estimated constituting the equipment. In a simple manner, the corrosion amount can be measured by burying the same kind of metal near the ground surface and measuring a weight change or a thickness reduction amount after a lapse of a certain period of time. Further, for example, the measurement unit 403 using an electrode for AC impedance including the same kind of metal as a sensor can be used.

In a simple manner, the same metal as a constituent metal of the equipment 401 is embedded near the ground surface, and the corrosion amount of the embedded metal is measured by the measurement unit 403 after a lapse of a certain period of time, whereby the corrosion amount and the corrosion rate can be acquired. In addition, for example, by burying an electrode for electrochemical measurement as a sensor on the ground surface, the corrosion amount and the corrosion rate can be acquired. As the electrochemical measurement, an AC impedance method is preferable. By using the same metal as the constituent metal of the equipment for the electrode and measuring a response to the alternating current, information on the corrosion rate can be obtained. In addition, the corrosion rate and the corrosion amount can be acquired by measuring a thickness reduction amount due to corrosion with a measuring instrument such as a caliper.

Furthermore, the corrosion estimation device 100 can be a computer device as described above, and can be implemented by, for example, a general personal computer or an electronic device such as a tablet. For example, the measurement unit 403 may be provided with a transmission function 404 for transmitting measured information so as to be able to communicate with the corrosion estimation device 100 via a communication network 405. In addition, one corrosion estimation device 100 can correspond to the plurality of measurement units 403. The display unit 105 can be implemented by a monitor of a personal computer, a wireless device, or the like.

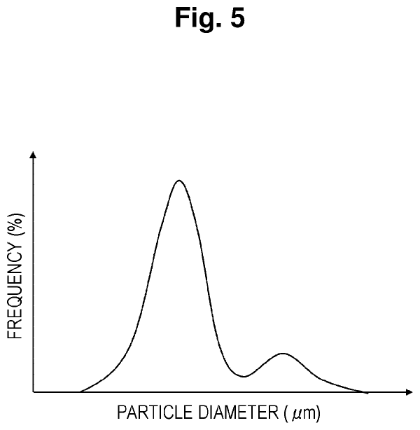

The particle diameter of the soil can be obtained by measuring the soil collected in the target land using a known particle diameter measuring device. The particle diameter of the soil can be acquired as a distribution as illustrated in FIG. 5. In addition, as the particle size of the soil, an average particle diameter can be simply used.

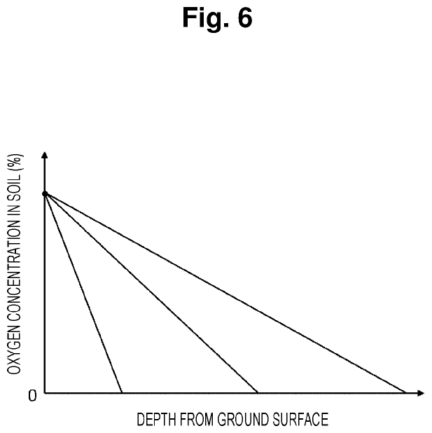

An example of the relationship between the underground depth and the oxygen concentration in the soil is illustrated in FIG. 6. Based on the idea of oxygen diffusion from the atmosphere near the ground surface into the soil, the above-described relationship can be obtained by estimating how the oxygen concentration in the soil changes with respect to the underground depth. For example, assuming that the oxygen concentration on the ground surface is the same value as the oxygen concentration in the atmosphere and the oxygen concentration at a sufficiently deep position is zero, the above-described relationship can be obtained by estimating with a model in which the concentration linearly changes with respect to the depth. The inclination of the straight line is related to the soil particle diameter. For example, the relationship between the soil particle diameter and an inclination of the concentration change with respect to the depth can be obtained in advance by an experiment or the like.

In addition, the relationship between the underground depth and the oxygen concentration in the soil can be determined (acquired) according to the following idea. Oxygen in the soil diffuses through voids in the soil. Therefore, it is considered that the ease of diffusion is related to the ratio of voids in a certain plane in the soil and a pseudo extension degree of a diffusion distance by passing through the voids. Accordingly, for example, on the assumption that the particles are arranged in a close-packed state from the soil particle diameter, the ratio of voids in the plane in the soil and the extension degree of the diffusion distance are calculated. From the calculated degree, the relationship of the inclination of the oxygen concentration with respect to the underground depth can be calculated with reference to diffusion in the atmosphere. In this manner, if the relationship between the soil particle diameter and the oxygen concentration with respect to the underground depth is modeled in advance, the state of the oxygen concentration with respect to the underground depth of the target ground can be calculated (acquired) from the obtained particle diameter.

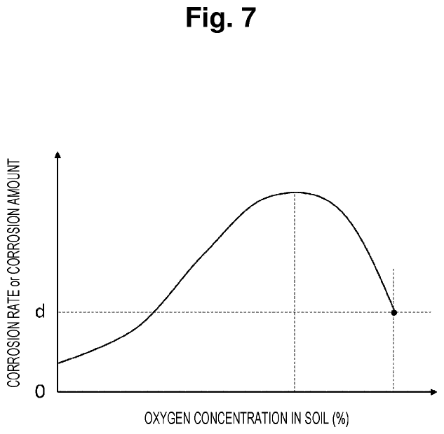

Next, the relationship between the oxygen concentration and the corrosion rate or the corrosion amount will be described with reference to FIG. 7. FIG. 7 illustrates an example of the relationship between the oxygen concentration and the corrosion rate or the corrosion amount. This relationship can be obtained in advance by an experiment or the like. In general soil such as black soil and red soil, it is known that there is a curve relationship having a maximum at about 10 to 18% with respect to the oxygen concentration in the soil. If the above-described relationship is obtained in consideration of the influence of the type of soil, the temperature, and the like, the estimation accuracy becomes higher.

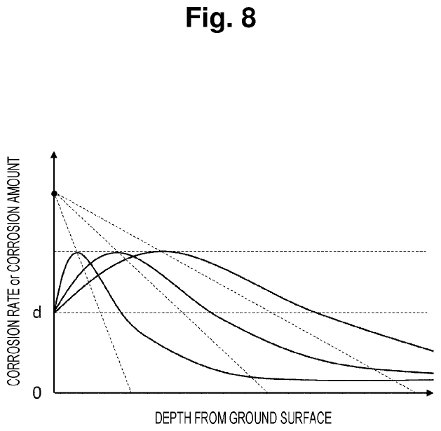

Next, the relationship between the underground depth and the corrosion rate or the corrosion amount will be described with reference to FIG. 8. FIG. 8 illustrates an example of the relationship between the underground depth and the corrosion rate or the corrosion amount. In FIG. 8, “d” is the corrosion rate or the corrosion amount of the target metal (structure) near the ground surface of the land. From the value of d, the relationship between the underground depth and the oxygen concentration in the soil, and the relationship between the oxygen concentration and the corrosion rate or the corrosion amount, the relationship between the underground depth and the corrosion rate or the corrosion amount can be obtained. Therefore, the corrosion rate or the corrosion amount with respect to the underground depth can be known only by acquiring the corrosion rate or the corrosion amount near the ground surface, so that it is possible to estimate the corrosion state of the deep portion from the ground surface.

As described above, according to embodiments of the present invention, the relationship between the underground depth and the corrosion rate or the corrosion amount in the land is estimated from the relationship between the oxygen concentration and the corrosion rate or the corrosion amount on the basis of the relationship between the underground depth of the land and the oxygen concentration in the soil and the corrosion rate or the corrosion amount of the target metal near the ground surface of the land. Thus, it is possible to easily and inexpensively inspect the deterioration state of the underground equipment made of metal.

Note that the present invention is not limited to the embodiments described above, and it is obvious that many modifications and combinations can be implemented by those skilled in the art within a technical scope of the present invention.

### REFERENCE SIGNS LIST

101 First estimation function unit
102 Second estimation function unit
103 Third estimation function unit
104 Storage unit
105 Display unit

1-4. (canceled)

5. A corrosion estimation method, comprising:
estimating, based a particle diameter of soil of a target land, a relationship between an underground depth of the target land and an oxygen concentration in soil; and
estimating, based a corrosion rate of a target metal at a ground surface of the target land and the relationship between the underground depth and the oxygen concentration, a relationship between the underground depth and the corrosion rate from a relationship between the oxygen concentration and the corrosion rate.

6. The corrosion estimation method according to claim 5, further comprising:
estimating a corrosion state of a structure constituted of the target metal from the relationship between the underground depth and the corrosion rate.

7. The corrosion estimation method according to claim 6, wherein the structure is buried in the target land.

8. A corrosion estimation device, comprising:
a central processing unit; and
a storage device storing a program to be executed by the central processing unit, the program including instructions to:
estimate, based a particle diameter of soil of a target land, a relationship between an underground depth of the target land and an oxygen concentration in soil; and
estimate, based a corrosion rate or a corrosion amount of a target metal at a ground surface of the target land and the relationship between the underground depth and the oxygen concentration, a relationship between the underground depth and the corrosion rate or the corrosion amount from a relationship between the oxygen concentration and the corrosion rate or the corrosion amount.

9. The corrosion estimation device according to claim 8, wherein the program includes further instructions to:
estimate a corrosion state of a structure constituted of the target metal from the relationship between the underground depth and the corrosion rate or the corrosion amount.

10. The corrosion estimation device according to claim 9, wherein the structure is buried in the target land.

11. The corrosion estimation device according to claim 8, wherein the instructions to estimate, based the corrosion rate or the corrosion amount of the target metal at the ground surface of the target land and the relationship between the underground depth and the oxygen concentration, the relationship between the underground depth and the corrosion rate or the corrosion amount from a relationship between the oxygen concentration and the corrosion rate or the corrosion amount comprises instructions to:
estimate, based the corrosion rate of the target metal at the ground surface of the target land and the relationship between the underground depth and the oxygen concentration, the relationship between the underground depth and the corrosion rate from a relationship between the oxygen concentration and the corrosion rate.

12. The corrosion estimation device according to claim 8, wherein the instructions to estimate, based the corrosion rate or the corrosion amount of the target metal at the ground surface of the target land and the relationship between the underground depth and the oxygen concentration, the relationship between the underground depth and the corrosion rate or the corrosion amount from a relationship between the oxygen concentration and the corrosion rate or the corrosion amount comprises instructions to:
estimate, based the corrosion amount of the target metal at the ground surface of the target land and the relationship between the underground depth and the oxygen concentration, the relationship between the underground depth and the corrosion amount from a relationship between the oxygen concentration and the corrosion amount.

13. A corrosion estimation method, comprising:
estimating, based a particle diameter of soil of a target land, a relationship between an underground depth of the target land and an oxygen concentration in soil; and
estimating, based a corrosion amount of a target metal at a ground surface of the target land and the relationship between the underground depth and the oxygen concentration, a relationship between the underground depth and the corrosion amount from a relationship between the oxygen concentration and the corrosion amount.

14. The corrosion estimation method according to claim 13, further comprising:
estimating a corrosion state of a structure constituted of the target metal from the relationship between the underground depth and the corrosion amount.

15. The corrosion estimation method according to claim 14, wherein the structure is buried in the target land.
